(19) United States
(12) Patent Application Publication
(10) Pub. No.: US 2005/0159717 A1
(43) Pub. Date: Jul. 21, 2005

(54) Bag for collecting body fluids provided with a device for opening and closing a discharge channel

(75) Inventor: Henri Holtermann, Saint-Jean-De-Luz (FR)

Correspondence Address: YOUNG & THOMPSON, 745 SOUTH 23RD STREET, 2ND FLOOR, ARLINGTON, VA, 22202, US

(73) Assignee: B. Braun Medical SAS, Boulogne-Billancourt (FR)

(21) Appl. No.: 10/514,750

(22) PCT Filed: May 20, 2003
(86) PCT No.: PCT/FR03/01526

(30) Foreign Application Priority Data
May 21, 2002 (FR) 02/06159

(51) Int. Cl. A61F 5/44

(52) U.S. Cl. 604/332

(57) ABSTRACT

The invention concerns a bag (1) for collecting body fluids, such as excrement, for a person having undergone an ostomy. The bag comprise a receptacle pouch (10) terminated by a neck (15), a discharge channel (20) linked to said neck (15) and having a waste opening (25), an opening/closing device to shift the discharge channel (20) from a folded closed state to an unfolded opened state and comprising first (32) and second (36) opening/closing means arranged transversely relative to the discharge channel (20). A film (11; 12) of the receptacle pouch (10) is provided externally with a rigidifying member (40) proximate the neck (15) and the second opening/closing means (36) of the discharge channel (20) is arranged, at least in folded closed state, in a slot (45) located between the rigidifying member (40) and said film (11; 12) of the receptacle pouch (10).

The invention relates to a bag for collecting body fluids provided with a device for opening and closing a discharge channel for discharging said body fluids outside the bag.

### FIELD OF THE INVENTION

After a surgical ostomy operation performed as a result of a malformation, tumour or inflammation, and in particular after an ileostomy or a colostomy, it is advisable to connect the ileum of the small intestine or the colon to the abdominal wall in order to re-establish the faecal material elimination functions which can no longer be performed automatically and which thus occur uncontrollably as digestion proceeds. A comfortable, hygienic device which does not disturb the patient's activities, such as work, sport or leisure, is thus provided to this end.

Consequently, the patient with a stoma is generally provided with a bag for collecting body fluids, which is set in place immediately after the operation, generally around an abdominal orifice for discharge of faecal material. This bag, which conventionally takes the form of a pouch of flexible woven material impermeable to the fluid which it contains, may be removable or its contents may be drained. In this particular embodiment, it is common to use a drainage device connected at the bottom of the bag to a narrower neck, such that the faeces flow out principally by gravity when the drainage device is in the open position.

### BACKGROUND OF THE INVENTION

Various types of drainage device for bags for collecting body fluids have been described in prior publications, and in particular in patent document U.S. Pat. No. 3,690,320. This patent describes a drainage device in which the narrower portion of the bag, or discharge channel, is divided into three parts separated from one another by two folds and held in position by strips of hook and loop material, such as “Velcro”. Opening takes place as soon as the strips are detached, and it is thus not very convenient or reliable because the strips may become detached by themselves, in particular if they get caught on clothes.

It is also already known from patent document U.S. Pat. No. 3,825,005 to provide a drainage device in which the opening and closing operations take place in two successive stages with an intermediate position in which it is necessary to provide a first closing means when a second means is opened, in order to prevent untimely opening and leaks.

However, in this device, keeping the first closing means closed in the intermediate position of the drainage device requires mechanical reinforcement of the films constituting the discharge channel, in order to withstand the pressure exerted on a fold forming a closure zone in which liquid stools to be discharged accumulate, reinforcing panels being used on the discharge channel. These panels are awkward for the patient wearing the bag, make the drainage device more complex to produce and may even impair the reliability of the leakproof nature of the drainage device in certain positions which the patient may adopt.

Document WO 01 28 470 describes an ostomy bag comprising a tongue fixed to a wall of the bag, which tongue folds back against a folded discharge channel.

Patent document U.S. Pat. No. 4,233,977 describes an ostomy bag having a relatively rigid strip which defines with one side of the bag a receptacle into which is introduced a folded discharge channel.

### SUMMARY OF THE INVENTION

The object of the invention is thus to eliminate the disadvantages listed above.

To do this, the invention proposes providing a drainage device in which the opening and closing operations are performed in two successive stages, with an intermediate position in which one of the portions of the discharge channel is held in a folded state, and the discharge channel lacks any reinforcement.

More precisely, the invention relates to a bag for collecting body fluids, such as faecal material, designed to be disposed on the abdomen of a patient having undergone an ostomy and comprising:
a fluid-tight pouch formed of two films joined together at their periphery and ending in a neck,
an elongate discharge channel connected to said neck and formed of two walls joined together longitudinally in fluid-tight manner, the discharge channel having a drainage orifice situated at the opposite end from the neck for body fluids to flow out of the pouch, and
an opening/closing device for transferring the discharge channel from a folded closed state to an unfolded open state, the opening/closing device comprising a first and a second opening/closing means provided transversely relative to the discharge channel,
in which said opening/closing means are both active when the discharge channel is in the closed state and said opening/closing device is adapted to open the discharge channel by operation, in two successive stages, of the first and then the second opening/closing means, one of the pouch films is provided on the outside with a stiffening member disposed in the vicinity of the neck, and the second opening/closing means of the discharge channel, at least in its folded, closed state, is disposed at least in part in a slot situated between the stiffening member and the pouch film.

In this bag, since said opening/closing means are both active when the discharge channel is in the closed state and said opening/closing device is adapted to open the discharge channel by operation, in two successive stages, of the first and then the second opening/closing means, one of the pouch films is provided on the outside with a stiffening member disposed in the vicinity of the neck and the second opening/closing means of the discharge channel is disposed, at least in its folded, closed state, in a slot situated between the stiffening member and the pouch film.

According to various preferred embodiments of the invention:
the stiffening member is substantially perpendicular to the discharge channel and takes the form of a loop of stiffer plastics material than the material constituting the films of the pouch and of the walls of the discharge channel,
the second opening/closing means comprises at least one transverse fold of the discharge channel sealing the latter when the opening/closing device is in the closed position and, moreover, a second fold disposed at the level of the neck, next to the stiffening member, whatever the open or closed position of the opening/closing device,
it comprises a handling tongue fixed to the second fold of the second opening/closing means, which passes through the slot situated between the stiffening member and the bag film and which extends beyond this member so as to be able to be gripped, such that the second opening/closing means may be displaced from its open-position to its closed position by pulling on the tongue,
the first opening/closing means is disposed, in the closed position, over the stiffening member and it comprises a self-gripping strip, having at least a first part, for example a male or female strip of the “Velcro” type or any type of repositionable adhesive, disposed on the handling tongue situated substantially at the opposite end from the drainage orifice of the discharge channel;
the handling tongue is connected to a fold of the second opening/closing means, and
the stiffening member has an extension on the opposite side of the opening of the slot situated between the stiffening member and the bag film on the bag neck side, virtually at the point of interaction of the parts of the self-gripping strip, and/or
the discharge channel is provided, away from the opening/closing means, with transverse folds facilitating folding and unfolding thereof.

### BRIEF DESCRIPTION OF THE DRAWINGS

Other features, details and advantages of the invention will become clear from a reading of the description made with reference to the attached drawings provided by way of example and in which:

FIG. 1 is a front view of a bag for collecting faecal material according to the invention and provided with a discharge channel in a totally closed state,

FIG. 2 is a side view of FIG. 1;

FIG. 3 shows a detail of FIG. 1, showing the first and second means of closing the discharge channel in a totally closed position;

FIG. 4 is a front view of the bag with the first closing means in an open, unfolded position;

FIG. 5 is a side view of FIG. 4;

FIG. 6 is a front view of the bag with the discharge channel in the intermediate opening position;

FIG. 7 is a side view of FIG. 6,

FIG. 8 is a front view of the bag with the discharge channel in a totally unfolded position, that is to say with the second closing means open,

FIG. 9 is a side view of FIG. 8,

FIG. 10 is a view similar to that of FIG. 8, but showing a variant, and

FIGS. 11 and 12 are diagrams indicating the positions of the various elements of the variant of FIG. 10.

### DETAILED DESCRIPTION OF THE PREFERRED EMBODIMENTS

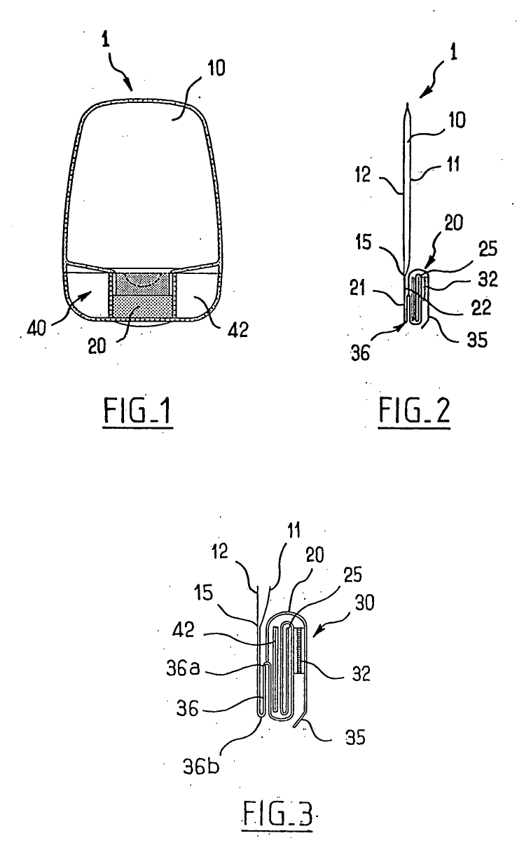

FIGS. 1 to 3 show, in the closed state, a bag 1 for collecting fluid faecal material flowing from an orifice (not shown) formed in the abdomen of a patient with a stoma following an ileostomy, for example.

This bag 1 comprises a pouch 10 consisting of two spaced thin films 11 and 12 welded together at their periphery so as to be leakproof relative to the faecal material. The pouch 10 also comprises an open neck 15 extended by an elongate channel 20 for discharging the faecal material out of the pouch 10 when it is necessary to drain the collecting bag 1.

The discharge channel 20 consists of two films made of the same material as those constituting the pouch, typically films providing an odour barrier, such as for example composite films sold under the trade name “Saranex”. Any other film made from polyethylene, PVC (polyvinyl chloride), PVDC (polyvinylidene chloride), EVA (ethylene vinyl acetate resin), or any polymer which may be extruded into a film, may also be suitable. These films form two spaced walls 21 and 22 welded longitudinally to one another by their respective edges to render them leakproof relative to the faecal material. The discharge channel 20 ends, at the opposite end from the neck 15, in a drainage orifice 25 allowing passage of the faecal material during draining of the bag 1.

The bag 1 is provided with a device 30 for opening/closing the discharge channel 20, said device 30 being actuatable by the user provided with said bag 1 so that he/she can drain it very easily on his/her own. To this end, the opening/closing device 30 comprises a first means 32 for opening/closing the discharge channel 20. In this example, this first opening/closing means 32 takes the form of a self-gripping strip sold under the registered trademark “Velcro”, a male part 33 of which is disposed on the inner face of a handling tongue 35 situated, as illustrated in FIGS. 6 to 9, substantially at the opposite end from the drainage orifice 25 of the discharge channel 20, when said channel 20 is in the unfolded, open state. The other, female part 34 of the first opening/closing means 32 is disposed on an outer surface of a wall 22 of the discharge channel, transversely relative thereto.

The opening/closing device 30 also comprises a second opening/closing means 36 formed by a first fold and a second fold 36a and 36b, disposed transversely relative to the discharge channel 20. The tongue 35 is connected to the first fold 36a, which is furthest from the neck 15 of the pouch 10, while the second fold 36b is disposed nearer to the neck 15.

The pouch 10 of the bag 1 is further provided with a stiffening member 40 in the vicinity of the neck 15. This member takes the form of a loop 42, similar to a belt loop, welded and/or glued externally to the film 11 of the pouch 10 over substantially the entire width of said pouch 10. Said loop 42 is made of a sturdier plastics material than the material constituting the pouch 10 and the discharge channel 20, taking the form for example of a polyethylene sheet, of a composite film of PE/PET (polyethylene/ polyethylene terephthalate), a film of rigid PVC or a PET film. Said loop 42 defines, with the film 11, a slot 45 perpendicular to the discharge channel 20 and in which there is disposed the second opening/closing means 36 when the opening/closing device 30 is in the closed, folded state, as is visible in the detail view in FIG. 3.

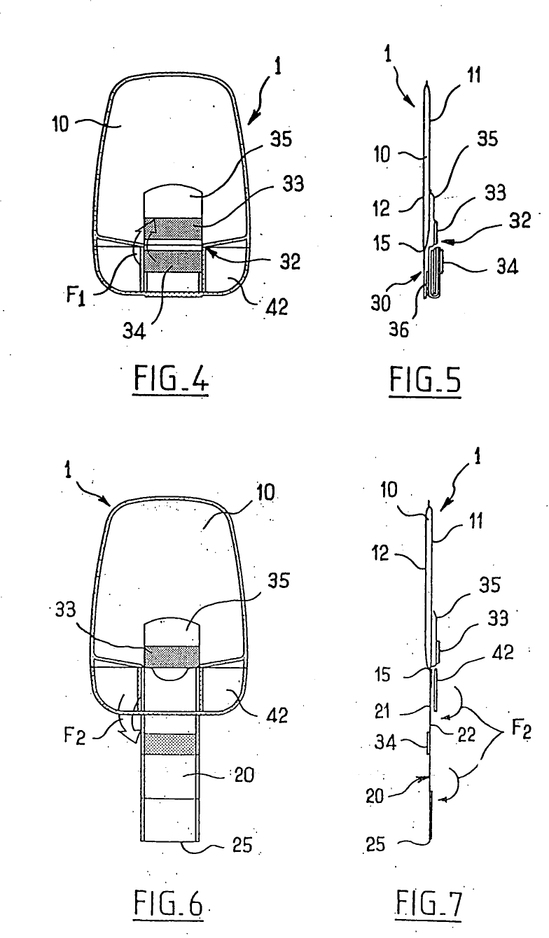

FIGS. 4 to 7 illustrate a first stage in the opening of the discharge channel 20. This first stage consists in detaching the strip of the first opening/closing means 32, which is located, in its closed state, over the loop 42. To do this, the user lifts the handling tongue 35, as illustrated by arrow F1 in FIG. 4. The result of this operation is illustrated by FIGS. 4 and 5, in which the strip 32 is shown in its open state.

Following this operation, the discharge channel 20 may be unfolded by the user into an intermediate position shown in FIGS. 6 and 7 and by the arrow F2. In this intermediate position, the discharge channel 20 is still kept closed by the second opening/closing means 36, the folds 36a and 36b of which are held in position by the loop 42.

All that is then needed is to pull lightly on the end of the discharge channel 20, preferably close to the drainage orifice 25, to unfold the accordion-type folds 36a and 36b disposed under the loop 42, in order to allow passage of the faecal material held back by these folds such that it flows into the discharge channel 20 and exits via the drainage orifice 25. In this open, unfolded position, the handling tongue 35 is likewise under the loop but remains accessible.

To close the discharge channel 20 again, all that is needed is to pull on the tongue to fold the second opening/closing means 36 back up, so that the folds 36a and 36b return to their accordion-folded position under the loop 42.

Then, the channel is folded back up in such a way as to position the male and female parts 33 and 34 of the first opening/closing means 32 opposite one another so as to close fully the discharge channel 20. To this end, the discharge channel 20 is provided, away from the opening/closing means 32 and 36, with transverse folds 26, 27 and 28 facilitating folding and unfolding thereof.

The bag for collecting body fluid 1 according to the present invention thus achieves a high level of security with regard to the leakproof nature of the discharge channel 2Q, even when the latter is partially open, and considerable ease of use, owing to the use of the stiffening loop 42 which holds the second opening/closing means 36 in place.

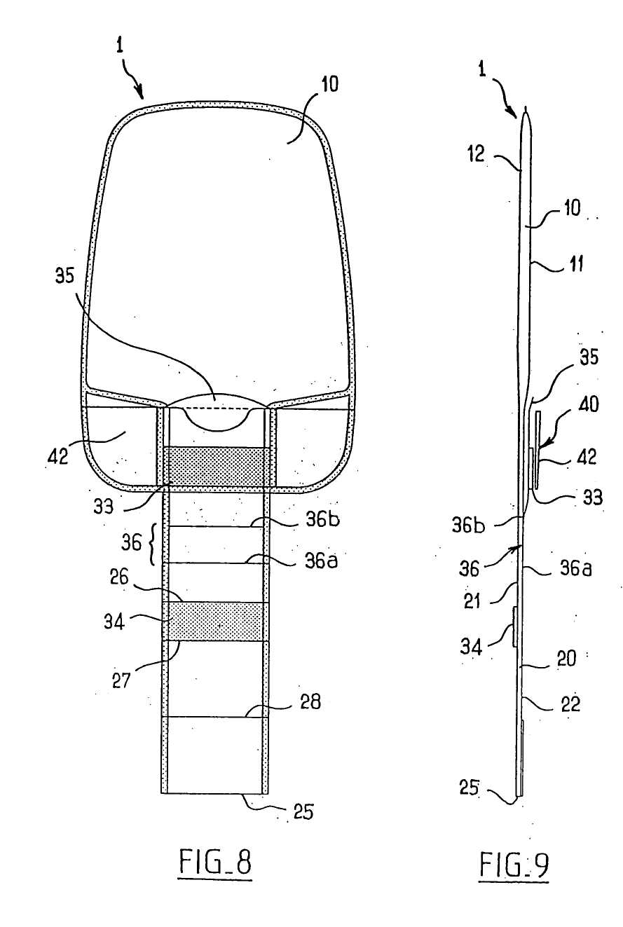

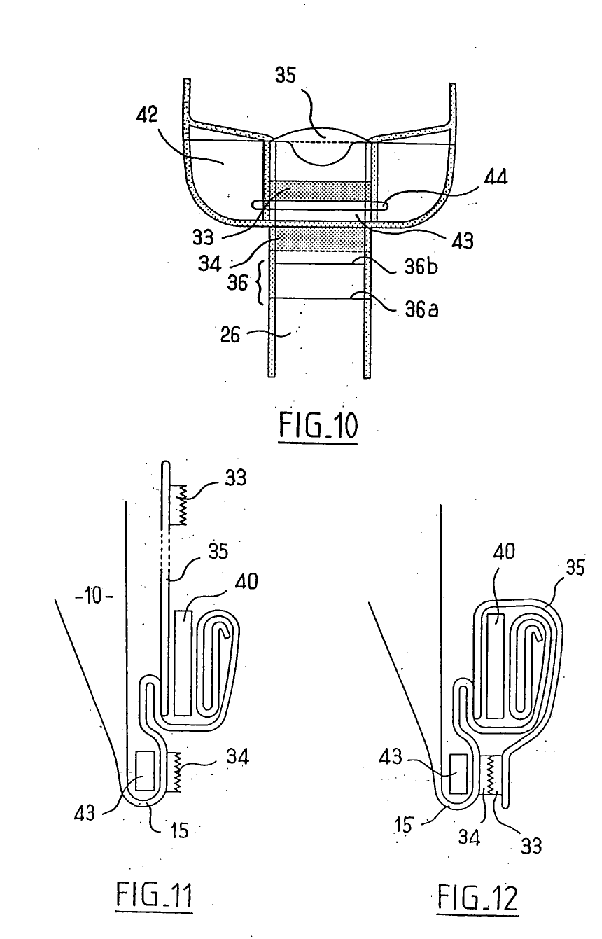

FIGS. 10 to 12 show a variant exhibiting different features from those of the embodiment of FIGS. 8 and 9.

In this embodiment, the self-gripping strip part 34 is above the fold 36b to which the tongue 35 is welded, that is to say closer to the neck 15. The self-gripping strip part 33 fixed to the tongue 35 surrounds the discharge channel more fully and holds it more firmly than in the embodiment of FIGS. 1 to 3, as shown in particular by a comparison of FIGS. 3 and 12.

An extension 43 of the stiffening member 40 is also shown, separated from the main part of this member by a slot 44 through which pass the folded discharge channel and the tongue. This extension constitutes a rigid support which supports the self-gripping strip parts when they are applied to one another on closure of the first opening/closing means.

It goes without saying, however, that these examples are given solely to illustrate the subject matter of the invention, which they do not limit in any way. In particular, said bag may be used for colostomies. The “Velcro” type strip may be replaced by a strip provided with a repositionable adhesive. The stiffening means 42 may take the form of a plurality of small, slightly spaced, rigid strips, in order to make the bag more comfortable to use.

1. A bag (1) for collecting body fluids, such as faecal material, designed to be disposed on the abdomen of a patient having undergone an ostomy and comprising:
a fluid-tight pouch (10) formed of two films (11, 12) joined together at their periphery and ending in a neck (15),
an elongate discharge channel (20) connected to said neck (15) and formed of two walls (21, 22) joined together longitudinally in fluid-tight manner, the discharge channel (20) having a drainage orifice (25) situated at the opposite end from the neck (15) for body fluids to flow out of the pouch (10), and
an opening/closing device (30) for transferring the discharge channel (20) from a folded closed state to an unfolded open state, the opening/closing device (30) comprising a first (32) and a second (36) opening/closing means provided transversely relative to the discharge channel (20),
characterised in that said opening/closing means (32, 36) are both active when the discharge channel (20) is in the closed state and said opening/closing device (30) is adapted to open the discharge channel (20) by operation, in two successive stages, of the first (32) and then the second (36) opening/closing means, one of the films (11; 12) constituting the pouch (10) is provided on the outside with a stiffening member (40) disposed in the vicinity of the neck (15), and the second opening/closing means (36) of the discharge channel (20), at least in its folded, closed state, is disposed at least in part in a slot (45) situated between the stiffening member (40) and the pouch film (11; 12).

2. A bag according to claim 1, characterised in that the stiffening member (40) is substantially perpendicular to the discharge channel (20).

3. A bag according to claim 1, characterised in that the stiffening member (40) takes the form of a loop (42) of stiffer plastics material than the material constituting the films (11; 12) of the pouch and of the walls (21, 22) of the discharge channel (20).

4. A bag according to claim 1, characterised in that the second opening/closing means (36) comprises at least one fold (36a) disposed transversely relative to the discharge channel (20) and sealing the latter when the opening/closing device (30) is in the closed position.

5. A bag according to claim 4, characterised in that the second opening/closing means (36) comprises a second fold (36b) disposed nearer to the neck (15) and the stiffening member than the first fold (36a), whatever the open or closed position of the opening/closing device (30).

6. A bag according to claim 1, characterised in that the first opening/closing means (32) is disposed, in the closed position, over the stiffening member (40).

7. A bag according claim 1, characterised in that it comprises a handling tongue (35) fixed to the second fold of the second opening/closing means (36), which passes through the slot situated between the stiffening member (40) and the film (11; 12) of the bag (15) and which extends beyond this member so as to be able to be gripped, such that the second opening/closing means (36) may be displaced from its open position to its closed position by pulling on the tongue (35).

8. A bag according to claim 7, characterised in that the first opening/closing means (32) comprises a self-gripping strip having at least a first part (33; 34) disposed on the handling tongue (35) situated substantially at the opposite end from the drainage orifice (25) of the discharge channel (20).

9. A bag according to claim 8, characterised in that the stiffening member (40) has an extension (43) on the opposite side of the opening of the slot situated between the stiffening member (40) and the bag film (11; 12) on the side of the bag neck (15), virtually at the point of interaction of the parts of the self-gripping strip.

10. A bag according to claim 1, characterised in that the discharge channel (20) is provided, away from the opening/closing means (32, 36), with transverse folds (26, 27, 28) facilitating folding and unfolding thereof.

11. A bag according to claim 2, characterised in that the stiffening member (40) takes the form of a loop (42) of stiffer plastics material than the material constituting the films (11; 12) of the pouch and of the walls (21, 22) of the discharge channel (20).

12. A bag according to claim 2, characterised in that the second opening/closing means (36) comprises at least one fold (36a) disposed transversely relative to the discharge channel (20) and sealing the latter when the opening/closing device (30) is in the closed position.

13. A bag according to claim 3, characterised in that the second opening/closing means (36) comprises at least one fold (36a) disposed transversely relative to the discharge channel (20) and sealing the latter when the opening/closing device (30) is in the closed position.

14. A bag according to claim 2, characterised in that it comprises a handling tongue (35) fixed to the second fold of the second opening/closing means (36), which passes through the slot situated between the stiffening member (40) and the film (11; 12) of the bag (15) and which extends beyond this member so as to be able to be gripped, such that the second opening/closing means (36) may be displaced from its open position to its closed position by pulling on the tongue (35).

15. A bag according to claim 2, characterised in that the discharge channel (20) is provided, away from the opening/closing means (32, 36), with transverse folds (26, 27, 28) facilitating folding and unfolding thereof.
